(19) United States
(12) Patent Application Publication
(10) Pub. No.: US 2022/0039665 A1
(43) Pub. Date: Feb. 10, 2022

(54) JUGULAR VENOUS PRESSURE MEASUREMENT DEVICES

(71) Applicant: JRAS MEDICAL INC., St. John's (CA)

(72) Inventors: ANDREW J. SMITH, St. John's (CA); JASON D. ROBERTS, St. John's (CA); ANDREW M. L. SMITH, St. John's (CA); ROM KOUBI, St. John's (CA)

(73) Assignee: JRAS MEDICAL INC., St. John's, NL (CA)

(21) Appl. No.: 17/275,170

(22) PCT Filed: May 3, 2019
(86) PCT No.: PCT/CA2019/050590

§ 371 Date: Mar. 10, 2021

Related U.S. Application Data
(60) Provisional application No. 62/730,416, filed on Sep. 12, 2018.

(51) Int. Cl. A61B 5/021; A61B 5/00

(52) U.S. Cl. CPC A61B 5/021; A61B 2560/02; A61B 5/0059; A61B 5/6822

(57) ABSTRACT

The present disclosure provides a device for measuring jugular venous pressure of a patient. The device comprises a body defining a longitudinal enclosure and having a window along a length of the longitudinal enclosure to allow light to exit the longitudinal enclosure, and a beam generator comprising an array of light emitters. The beam generator is configured to direct light out the window to generate a beam of light along a plane perpendicular to a longitudinal direction and at an adjustable position along the longitudinal direction. The device has an adjustment mechanism for adjusting the position of the beam of light relative to the body along the longitudinal direction, and a readout indicating the position of the beam of light along the longitudinal direction.

### CROSS-REFERENCE TO RELATED APPLICATION

This application claims the benefit of priority of U.S. Provisional Patent Application No. 62/730,416, which was filed on Sep. 12, 2018 and is hereby incorporated herein by reference in its entirety.

### TECHNICAL FIELD

The present disclosure relates to devices for measuring the jugular venous pressure of a patient.

### BACKGROUND

Congestive heart failure (CHF) is a common and devastating health problem that affects upwards of 23 million individuals worldwide. Beyond incapacitating symptoms of shortness of breath and fatigue, long-term prognosis of CHF patients is extremely poor with only 50% and 10% of affected patients being alive at 5 and 10 years, respectively. Proper medical management of CHF is critical for improving symptoms and prolonging life and relies heavily on the physical examination. The primary goal of the physical exam among CHF patients is to evaluate for signs of volume overload, as excessive intravascular volume results in fluid backing up in the lungs causing shortness of breath and strain on the heart. Although there are multiple features that facilitate evaluation of volume status, the most informative is the jugular venous pressure (JVP). Assessment of the JVP involves attempting to visualize the height of a column of blood in a neck vein (internal jugular vein) just below the skin. Typically, the patient is placed in a semi-recumbent position, in the range of 30°-60° to the horizontal, with the head rotated away from the side being examined (10°-30° rotation). The clinician then examines the patient's neck to determine the height of the venous column demarked by the highest biphasic pulsation of the skin (as opposed to uniphasic pulsation of the adjacent carotid artery). Unfortunately, clinical assessment of the JVP is notoriously inaccurate and challenging to measure. This is a major clinical issue as optimal management of heart failure patients depends upon accurate assessment of the JVP. Inaccurate measurement may mislead clinical management decisions and result in adverse clinical outcomes.

Two major difficulties associated with measuring the JVP that can result in inaccurate measurements are: 1) Failing to correctly identify the height of the venous column of fluid along the neck, and 2) Ascertaining the height of the venous column relative to the sternal angle (a palpable landmark located along the chest at the level of the second ribs). The JVP is reported as height of the column of blood in the internal jugular vein, in centimeters, above the sternal angle with this value serving to guide subsequent medical therapy. An elevated JVP will generally trigger clinicians to diurese (remove fluid from) a patient in order to reduce volume overload, while a normal or low JVP reduces the likelihood that the patient is in active heart failure. A major challenge in ascertaining the correct height of the JVP relative to the sternal angle relates to the distance between the venous column in the neck and the sternal angle. Clinicians routinely make a visual estimation of the height, which is invariably error prone. More objective measurement of the JVP and standard training in medical school involves placing a ruler perpendicular to the horizontal plane and extending another straight edge from the ruler to the height of the venous column on the neck. This technique is cumbersome and difficult to perform. This is further compounded by clinicians rarely ever carrying two long rulers in their pocket during routine clinical rounds. As a result, this method is rarely ever performed in routine clinical practice.

Various devices have been proposed to facilitate measurement of the JVP, including (Patent US20100094141) and (Patent US20080294070). Neither of these techniques address the cumbersome features of the double ruler method, as both still involve extending a straight edge from a ruler aligned at the sternal angle.

The inventors have determined a need for improved devices for measuring the JVP.

### SUMMARY

One aspect provides a device for measuring jugular venous pressure of a patient. The device comprises a body defining a longitudinal enclosure and having a window along a length of the longitudinal enclosure to allow light to exit the longitudinal enclosure, and a beam generator comprising an array of light emitters. The beam generator is configured to direct light out the window to generate a beam of light along a plane perpendicular to a longitudinal direction and at an adjustable position along the longitudinal direction. The device has an adjustment mechanism for adjusting the position of the beam of light relative to the body along the longitudinal direction, and a readout indicating the position of the beam of light along the longitudinal direction. Some aspects also provide a level and/or a secondary light source integrated into the device.

Further aspects and details of example embodiments are set forth below.

### DRAWINGS

The following figures set forth embodiments in which like reference numerals denote like parts. Embodiments are illustrated by way of example and not by way of limitation in the accompanying figures.

FIG. 1 shows an example device for measuring JVP according to one embodiment of the present disclosure.

FIG. 2 is a longitudinal sectional view of the device of FIG. 1.

FIG. 2A shows a longitudinal sectional view of a portion of a device with a different beam generator and lens configuration according to another embodiment of the present disclosure.

FIG. 2B shows a longitudinal sectional view of a device for measuring JVP with an internal support rod according to another embodiment.

FIG. 2C shows a longitudinal sectional view of a portion of a device with the beam generator and lens configuration of FIG. 2A and the internal support rod of FIG. 2B.

FIG. 3 is a lateral sectional view of the device of FIG. 1.

FIG. 3A is a lateral sectional view of the device of FIG. 2B.

FIG. 4 shows the device of FIG. 1 projecting a light beam.

FIG. 5 shows a testing apparatus for the device of FIG. 1.

FIG. 5A shows the device of FIG. 1 with an adjusted scale applied thereto according to another embodiment of the present disclosure.

FIG. 6 shows another example device for measuring JVP according to one embodiment of the present disclosure.

FIG. 6A is an exploded view showing various components of the embodiment of the device shown in FIG. 6.

FIG. 6B shows an exploded view of a device similar to the FIG. 6 example with an alternative adjustment mechanism according to another embodiment.

FIG. 6C shows an exploded view of a device similar to the FIG. 6 example with an alternative adjustment mechanism according to another embodiment.

### DETAILED DESCRIPTION

The following describes an example embodiment of a device for measuring the JVP. The device has an elongated body which is oriented vertically when in use, and contains a beam generator that transmits a horizontal beam of light perpendicular to the vertical axis from an adjustable position along the body of the device. In some embodiments, the beam is projected directly onto a patient. In some embodiments, the horizontal beam of light passes through a lens to produce a sheet of light oriented along a substantially horizontal plane, and the sheet of light is projected onto the patient. In some embodiments, the horizontal beam of light may be formed into another shape that may be projected onto the patient and has a defining feature (e.g. an edge, a corner, or the like) at a height corresponding to the adjustable position along the body of the device.

In some embodiments, the vertical height of the light may be adjusted through adjustment of the height of a moveable portion of the beam generator within the device body. As discussed below, in some embodiments, the beam generator comprises a fixed light source and a moveable reflector, and in other embodiments the beam generator comprises a moveable light source, and in other embodiments the beam generator comprises an array of light emitters which may be selectively activated to generate the beam of light at an adjustable position. In some embodiments the moveable portion of the beam generator comprises a lens, and in other embodiments a lens may be fixed and incorporated into a window on the device body. In some embodiments, there may not be any lens, and the beam of light may be projected directly onto the patient.

The bottom edge of the device is designed to sit comfortably on the sternal angle of a patient inclined at a position approximately 45° (range: 30°-60°) from the vertical, with the device oriented vertically. The beam is then directed towards the side of the patient's neck (typically right) where the height of the jugular venous column can be visualized. The level of the horizontal sheet of light can then be adjusted to the height of the venous column by vertically adjusting the height of the moveable portion of the beam generator by means of an adjustment mechanism. When the beam is manually aligned with the height of the jugular venous column, the clinician simply reads the height (e.g. in cm) from a readout on the device. Manual vertical alignment may be assisted by detent stops or other tactile features. In some embodiments, the adjustment mechanism provides detent stops every 0.5 cm.

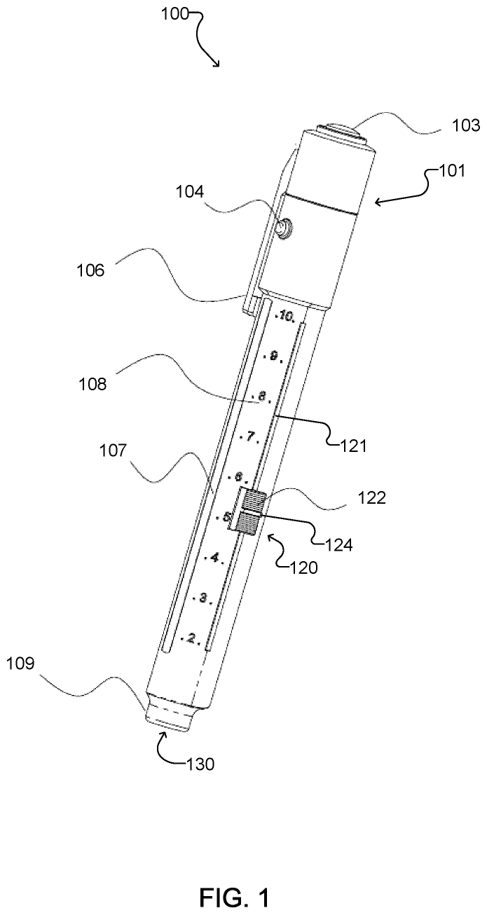

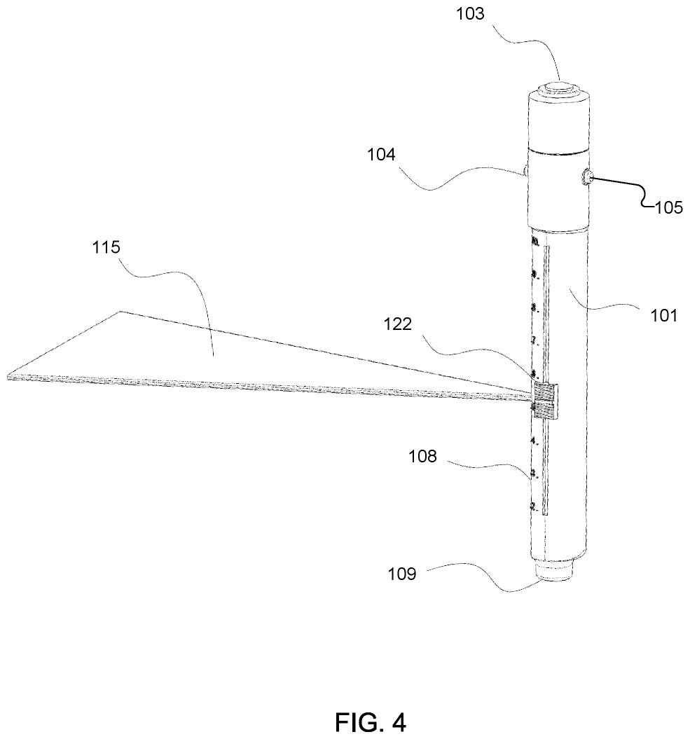

In the illustrated example of FIGS. 1-4, a button spirit level is provided at the top of the device body to enable the clinician to position the device vertically such that the beam is projected in a horizontal plane. In this example, the height of the horizontal sheet of light is adjusted using an adjustment mechanism in the form of a slider mechanism, and the readout comprises a scale next to the slider, as described further below. In other embodiments, the adjustment mechanism may comprise a different type of slider mechanism, a thumb wheel mechanism (e.g., a rack and pinion), a twisting or screw-type mechanism (e.g., twisting the base of the body to adjust the height of the sheet of light), one or more electromechanical switches connected to selectively activate one of a plurality of light emitters arranged in an array, or another suitable mechanism.

The example devices described below are ergonomically shaped and designed for use with either one or both hands. In some embodiments, the device also includes a second light source in the form of a broad spectrum light emitting diode (LED) (e.g. a “white” LED) integrated into the bottom of the device body to serve as a pen-light for a variety of other clinical assessments. In some embodiments the device may also include a pocket clip which may incorporate a switch for the LED.

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

FIGS. 1, 2, 3 and 4 show an example device 100 for measuring JVP. The device 100 comprises an elongated body 101 that defines a longitudinal enclosure 102. The body 101 has a level 103 thereon for ensuring that the body 101 is vertical when measuring JVP as discussed below. In the illustrated example, the level 103 is on the top of the body 101. A beam switch 104, secondary light switch 105, and pocket clip 106 are also provided on an upper portion of the body 101 in the illustrated example. The beam switch 104 is operable to activate a beam generator 110 as discussed below. The secondary light switch 105 is operable to activate a secondary light (e.g. an LED) 130 at a bottom end 109 of the body 101. The switches 104 and 105 may, for example, comprise momentary switches or toggle on/off switches. The location and configuration of the switches 104 and 105 may differ in other embodiments. In some embodiments the beam switch 104 and/or the secondary light switch 105 may, for example, be incorporated into the button spirit level 103 or the pocket clip 106, into the slider 122, or into a lower portion of the body 101.

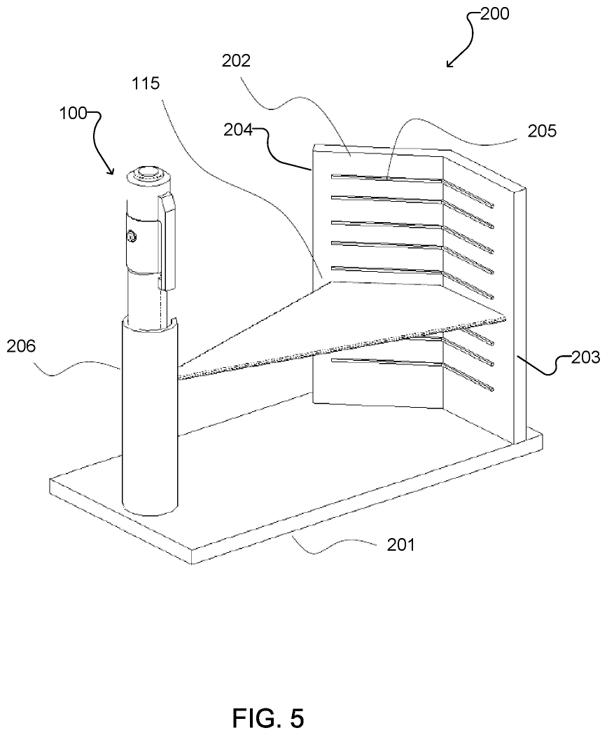

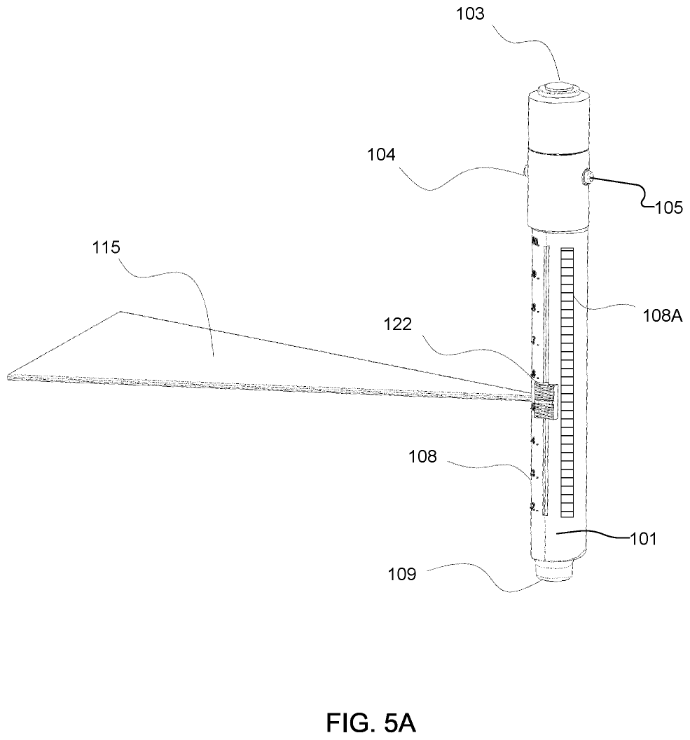

In the illustrated example of FIGS. 1-4, the beam generator 110 comprises a moveable portion adjustably mounted within the enclosure 102. The beam generator 110 is configured to generate a sheet of light 115 within a plane perpendicular to the longitudinal axis of device 100, as described further below, such that when device 100 is vertical, the sheet of light 115 is horizontal. The position of the moveable portion of the beam generator 110 within the enclosure can be adjusted by an adjustment mechanism 120. A window 107 is provided in the body along the length of the enclosure 102 to allow light to exit the body 101. A readout such as a scale 108 is provided on the body 101 for indicating the position of the beam generator 110 within the enclosure 102. In some embodiments, the scale 108 may be printed on the body after calibration of the device, or a corrected scale 108A may be adhered to the body 101, to compensate for any errors and accurately reflect the height of the sheet of light 115 at a distance of 15 cm away from the device 100, as discussed below with reference to FIGS. 5 and 5A.

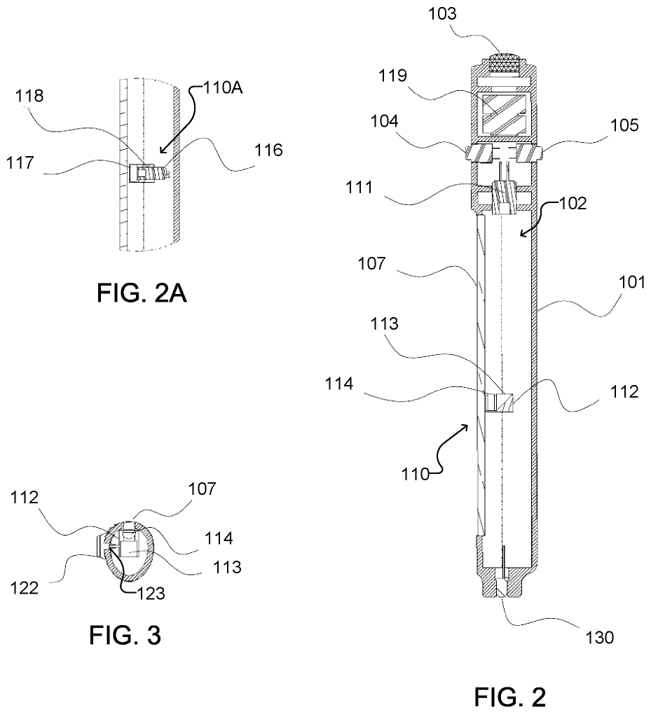

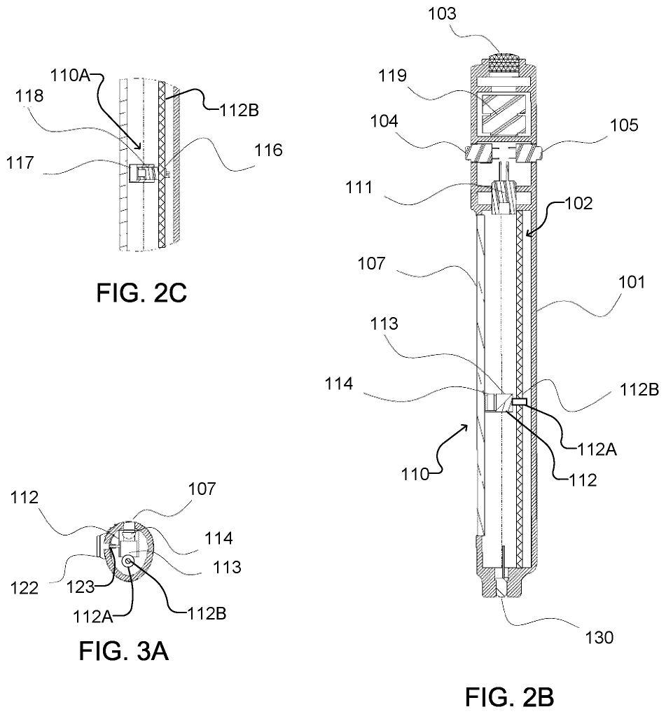

In the illustrated example, as best seen in FIG. 2 the beam generator 110 comprises a light source in the form of a laser 111 mounted in an upper portion of the body 101 above the enclosure 102. The moveable portion of the beam generator 110 comprises an optical assembly comprising a reflector 113 (e.g. a prism or mirror) and a lens 114, which are mounted on a platform 112 slidably mounted within the enclosure 102. A battery 119 is provided in the upper portion of the body 101 for powering the laser 111. In other embodiments, a laser or other light source could be mounted in a lower portion of the body 101 below the enclosure 102. In other embodiments, the lens 114 may be omitted, and the window 107 may comprise a lens to spread the light to generate the sheet 115. Other embodiments may have a beam generator 110A wherein the moveable portion comprises a laser or other light source 116 and lens 117 mounted on a slidable platform 118, as shown in FIG. 2A. In other embodiments, the moveable portion of the beam generator may comprise a light source mounted on a slidable platform with the window 107 functioning as a lens.

In the illustrated example, the adjustment mechanism 120 comprises a slider 122 connected to the platform 112 through a slot 121 in the body 101. The slot 121 is sealed with a flexible elastomer seal 123 configured to keep dust and contaminants out of the enclosure 102 while allowing movement of the slider 122. The slider 122 has an indicator mark 124 thereon adjacent to the scale 108. The slot 121 may have detent stops positioned periodically along its length, for example every 0.5 cm. The scale 108 and adjustment mechanism 120 are configured such that the indicator mark 124 is adjacent to a marking on the scale 108 indicating the height of the sheet of light 115 above the bottom end 109 of the body 101.

In some embodiments, the platform 112/118 is held in place by frictional bearing support from the edges of the body 101 around the slot 121. In other embodiments, one or more additional elements may provide support for the platform 112/118. For example, FIGS. 2B and 3A show an embodiment wherein a ring 112A attached to platform 112 slides along a supporting rod 112B extending longitudinally within the enclosure 102. The platform 112 could be coupled to the supporting rod 112B in other ways in other embodiments. For example, in some embodiments the platform 112 has an aperture therethrough sized to receive the supporting rod 112B such that the platform 112 can slide up and down the rod 112B. In some embodiments the platform 112 has a clip formed therein (e.g., a small ‘c’ integrated into its shape) and configured to engage the supporting rod 112B. As shown in FIG. 2C, the platform 118 of FIG. 2A could also be supported by a supporting rod 112B.

In operation, a clinician places the bottom 109 of the body 101 on a patient's sternal angle, and adjusts the position of the device to ensure the body 101 is vertical, as indicated by the level 103. The clinician then adjusts the height of the sheet of light 115 until it is aligned with the column of blood in the patient's vein, and reads the height from the scale 108.

FIG. 5 shows a testing apparatus 200 for testing the device 100. Apparatus 200 comprises a base 201, with a laser sight panel 202 comprising a perpendicular portion 203 and an angled portion 204 having gauge markings 205 thereon extending upwardly from the base 201. A sleeve 206 also extends upwardly from the base 201, and holds the device 100 perpendicularly to the base 201 such that the slider 122 is accessible and the scale 108 is visible. The perpendicular portion 203 and angled portion 204 are positioned at a predetermined distance to the sleeve 206 corresponding to a typical horizontal distance from the device to a patient's neck in a clinical setting (e.g. about 15 cm). A user can test the device 100 by inserting it on the sleeve 206 and activating the beam generator 110 to generate the sheet of light 115, then compare the height of the sheet of light 115 as measured by the gauge markings 204 with the height as indicated by the scale 108 on the device to ensure the heights match.

In some embodiments, the scale 108 may be printed on the body 101, or may be on a sticker or the like applied to the body 101, after calibration of the device 100 (for example by testing utilizing apparatus 200 or other testing apparatus) to account for any height mismatch. In some embodiments, a corrected scale 108A may be adhered to the body after testing, as shown in FIG. 5A.

The testing apparatus 200 is also useful for indicating any pitch or yaw angular errors in the orientation of the sheet of light 115. If the sheet of light 115 is not perpendicular to the device axis and ‘pitching’ up or down, this will result in a laser image line that is not parallel to the gauge markings 205 on the angled portion 204. Yaw angular errors are illustrated on the perpendicular portion 203 in a similar manner. If the sheet of light 115 is tipped (yaw) it will no longer be parallel on the surface of perpendicular portion 204 when compared to the markings 205. In some embodiments, the testing apparatus 200 also includes a mechanism for automatically activating the beam generator 110 when the device 100 is in the sleeve 206 (for example a physical feature attached to the sleeve 206 and positioned to contact the beam switch 104).

In some embodiments, the device 100 may be configured to interact with, or be incorporated into, other medical devices. For example, in some embodiments the device 100 includes a transducer or other type of sensor that generates a JVP signal based on the detected height, and a transmitter configured to send the JVP signal to another device such as an ultrasound or dialysis machine. In some embodiments, the device 100 transmits the detected height data to an ultrasound or dialysis machine via Bluetooth™ or other wireless transmission, or via wired transmission. In some embodiments, an ultrasound machine may be used to image the internal jugular vein (e.g. in long axis and/or transverse) and precisely determine the top of the column of fluid therein, which may be delineated on the patient's skin (either by the clinician visually identifying a feature on the skin at that height, or by applying a marking with, for example, a pen or marker). The device 100 may then be used as described above to determine the JVP height. In some embodiments, the device 100 may be incorporated into an ultrasound probe such that a single device can be used to image the internal jugular vein and determine the JVP height.

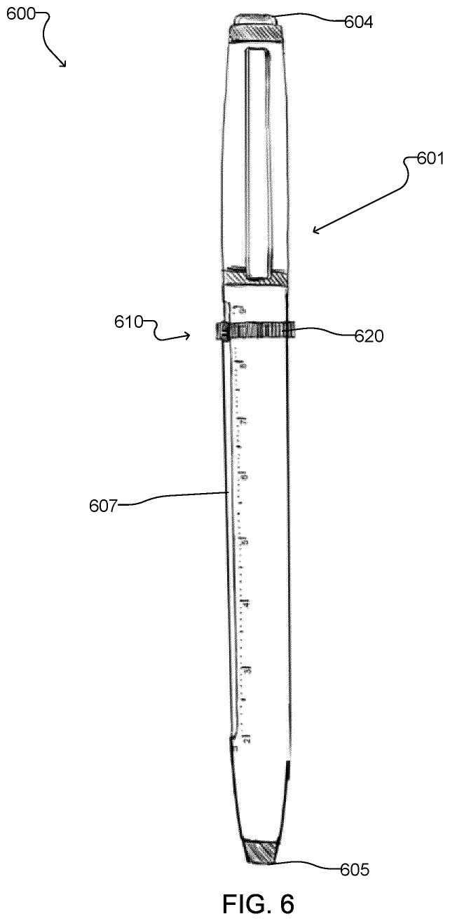

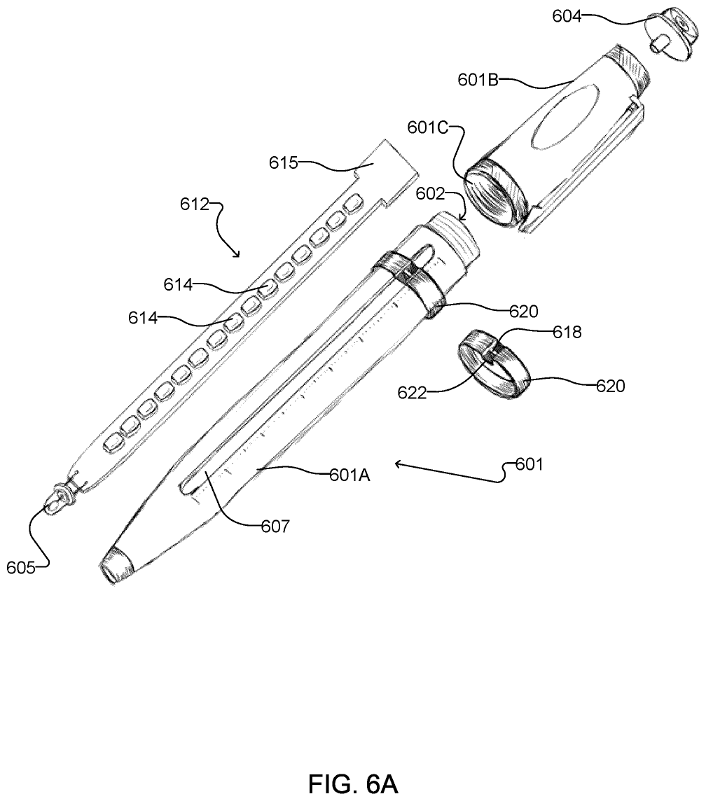

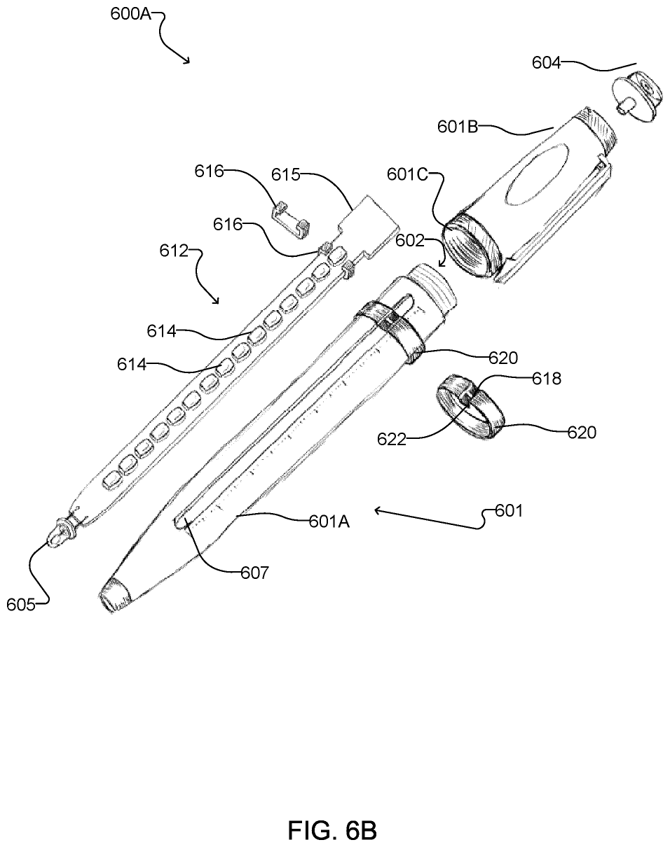

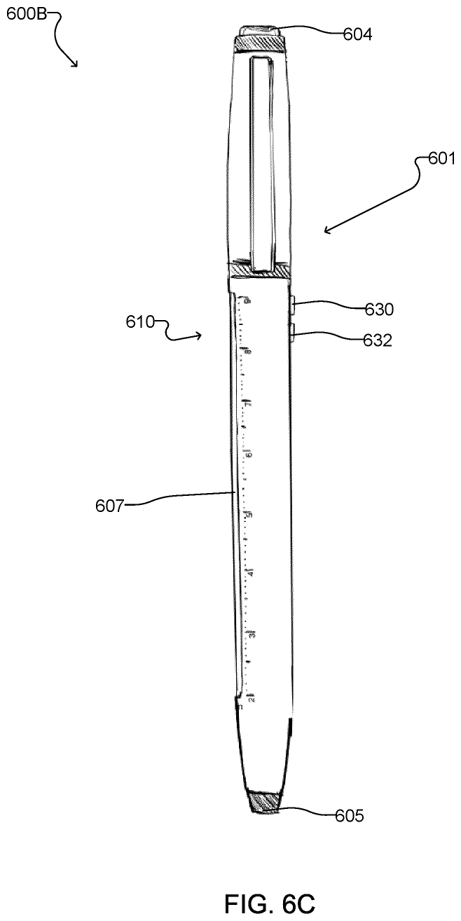

FIGS. 6 and 6A show another example device 600 for measuring JVP according to one embodiment of the present disclosure. In this embodiment the device 600 similarly comprises an elongated body 601 that defines a longitudinal enclosure 602 having a window 607. In this embodiment, the beam generator 610 comprises a fixed light source 612, comprising an array of light emitters mounted on a printed circuit board 615 situated within the longitudinal enclosure 602. In the illustrated example, the light emitters of the light source 612 comprise LEDs 614. In other embodiments, other types of light emitters (e.g. lasers) may be used. In some embodiments, the spacing between adjacent LEDs 614 is approximately 0.5 cm, but in other embodiments the spacing could be greater or less depending on the desired level of precision. In some embodiments, the moveable portion of the beam generator 610 comprises a lens 618 mounted on a slider 620, where the slider 620 is slidably mounted on the elongated body 601. The lens 618 may be on an indented portion 622 of the slider 620 that is in a recess within the elongated body 601 where the window 607 is located. The lens 618 spreads the light or generates the sheet 115 from the fixed light source 612. The position of the moveable portion of the beam generator 610 can be adjusted by moving the slider 620. The slider 620 is operably coupled to the fixed light source 612 such that when the lens 618 is aligned with one of the LEDs 614, that LED 614 turns on, as discussed below. In other embodiments, the beam generator 610 may have no moveable portion, and the position of the light may be adjusted by other means, such as for example by providing one or more electromechanical switches on the device for selectively activating light emitters of the array, as described below.

The device 600 may also comprise a level 604 and a “pen-light” 605. In some embodiments, the level 604 may also function as a switch to selectively activate the pen-light 605 (e.g. by a single click of a button integrated into the level 604) or the light source (e.g. by a double click of a button integrated into the level 604).

In the embodiment of FIGS. 6 and 6A, the elongated body 601 comprises a lower portion 601A and a cap portion 601B which may be held together by a threaded portion 601C. The threaded portion 601C may comprise a gasket, O-ring or other sealing mechanism for sealing the enclosure 602 to keep dust and contaminants out of the enclosure 602.

In the embodiment of FIGS. 6 and 6A, the slider 620 is shaped as a ring that slides over the body 601. In other embodiments the slider 620 may be a portion of a ring extending part way around the body 601, or may be an element configured to be slidingly received in a slot on the exterior of the elongated body 601. In other embodiments, the slider may be omitted.

In the embodiment of FIGS. 6 and 6A, the slider 620 comprises one or more magnets, and is coupled to the fixed light source 612 magnetically, such that the LED's 614 may be turned on without physical contact by the slider 620, thereby avoiding the need for any slot or other aperture into the enclosure 602. For example, the printed circuit board 615 may comprise a magnetically activated switch (for example, a reed switch) associated with each LED 614, and the slider 620 may have a magnetic portion that activates one of the switches on the printed circuit board 615 to turn on the associated LED 614. In this embodiment the device 600 may be completely enclosed to prevent contaminants from entering the longitudinal enclosure 602.

Alternatively, in other embodiments the printed circuit board 615 may have other types of sensors that do not require physical contact to activate the LEDs 614. For example, in some embodiments, a Hall effect sensor, or solid state transistors, may be associated with each LED 614 and configured to activate an LED 614 with the slider 620 is aligned therewith.

In some embodiments, the printed circuit board 615 comprises a physically activated switch (for example, a pair of contacts that must be electrically connected to complete a circuit) associated with each LED 614. For example, FIG. 6B shows an example device 600A, wherein the slider 620 is operatively coupled to an electrical conductor 616 within the enclosure 602. When the slider 620 moves, the conductor 616 moves along the printed circuit board 615 and completes a circuit to turn on one of the LEDs 614 when the slider is positioned over that LED 614.

In some embodiments, the slider may be omitted, and the printed circuit board 615 may be operatively coupled to an electronic control that a user can operate to selectively turn on any one of the LEDs 614. For example, FIG. 6C shows an example embodiment of a device 600B with an adjustment mechanism in the form of electromechanical switches. In the illustrated example, the device 600B comprises a pair of buttons 630 and 632 on the body, and the user can press the buttons 630/632 to change which LED 614 is turned on. For example, a user can press an “up” button 630 to turn off the currently active LED and turn on the LED immediately thereabove, and can press a “down” button 632 to turn off the currently active LED and turn on the LED immediately therebelow. In such embodiments, a lens may be integrated into the window 607, or each LED may have a lens associated therewith (e.g., integrated into the LED package or mounted on the printed circuit board).

In the embodiment of FIGS. 6 and 6A, the slider 620 comprises a lens 618 as discussed above. In other embodiments, the window 607 may comprise the lens for spreading the light and generating the sheet 115 from the fixed light source 612. In other embodiments each LED 614 has its own lens associated therewith. In such embodiments the beam generator 610 may comprise no moving parts and rather the position of the sheet 115 will depend on what portion of the light source 612 is illuminated. The portion of the light source that is illuminated may be controlled by buttons on the elongated body 601, for example, as discussed above with reference to FIG. 6B. In such embodiments the device 600 may be completely enclosed to prevent contaminants from entering the longitudinal enclosure 602.

In some embodiments, once turned on each LED 614 of light source 612 may remain on for a defined period before turning off (for example, for 15-20 seconds).

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing implementation of the various example embodiments described herein.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Although the embodiments have been described in detail, it should be understood that various changes, substitutions and alterations can be made herein. Moreover, the scope of the present application is not intended to be limited to the particular embodiments of the process, machine, manufacture, composition of matter, means, methods and steps described in the specification.

The present disclosure may be embodied in other specific forms without departing from its spirit or essential characteristics. The described embodiments are to be considered in all respects only as illustrative and not restrictive.

1. A device for measuring jugular venous pressure of a patient comprising:
a body defining a longitudinal enclosure and having a window along a length of the longitudinal enclosure to allow light to exit the longitudinal enclosure;
a beam generator comprising an array of light emitters, wherein the beam generator is configured to direct light out the window to generate a beam of light along a plane perpendicular to a longitudinal direction and at an adjustable position along the longitudinal direction;
an adjustment mechanism for adjusting the position of the beam of light relative to the body along the longitudinal direction; and,
a readout indicating the position of the beam of light along the longitudinal direction.

2. The device of claim 1 wherein the array of light emitters comprises an array of LEDs.

3. The device of claim 1 wherein the adjustment mechanism comprises one or more electromechanical switches control operatively coupled to the array of light emitters to selectively activate any one of the light emitters.

4. The device of claim 3 wherein the one or more electromechanical switches are configured to selectively activate the light emitters in a sequential fashion.

5. The device of claim 3 wherein the one or more electromechanical switches comprises an up button and a down button.

6. The device of claim 1 wherein the adjustment mechanism comprises a slider slidably attached to the body.

7. The device of claim 6 wherein each light emitter has a non-contact switch associated therewith configured to activate that light emitter when the slider is aligned with that light emitter.

8. The device of claim 7 wherein the slider comprises a magnetic portion and each light emitter has a magnetic switch associated therewith.

9. The device of claim 7 wherein the slider is coupled to an electrical conductor within the enclosure that selectively completes a circuit associated with each LED.

10. The device of claim 1 wherein the beam generator further comprises one or more lenses for generating a sheet of light along the plane perpendicular to the longitudinal direction.

11. The device of claim 10 wherein the one or more lenses are mounted on the adjustment mechanism.

12. The device of claim 10 wherein the one or more lenses are integrated into the window.

13. The device of claim 10 wherein each of the one or more lenses is associated with one of the light emitters.

14. The device of claim 1 wherein the light emitters of the array of light emitters are spaced at 0.5 centimeter intervals.

15. The device of claim 1 wherein the light emitters of the array of light emitters are spaced at 0.25 centimeter intervals.

16. The device of claim 1 wherein, once turned on, each of the one or more light emitters stays on for a defined period before turning off automatically.
